(19) United States
(12) Patent Application Publication
(10) Pub. No.: US 2001/0046684 A1
(43) Pub. Date: Nov. 29, 2001

(54) Methods of structure-based drug design using MS/MNR

(76) Inventors: Robert Powers, Westford, MA (US); Franklin J. Moy, Arlington, MA (US); Marshall M. Siegel, Fair Lawn, NJ (US); Dominick Mobilio, Warren, NJ (US)

Correspondence Address: Egon E. Berg, American Home Products Corporation, Patent Law Department - 2B, One Campus Drive, Parsippany, NJ, 07054, US

(21) Appl. No.: 09/789,345

(22) Filed: Feb. 21, 2001

Related U.S. Application Data
(60) Provisional application No. 60/287,579, filed on Feb. 25, 2000.

(51) Int. Cl. G01N 33/53; G06F 19/00

(52) U.S. Cl. 435/7.1; 702/19

(57) ABSTRACT

The present invention provides methods of structure-based drug design using mass spectrometry/NMR.

This application claims the benefit of U.S. patent application Ser. No. 09/513,806 and Provisional Application No. (Not Yet Known), filed Feb. 25, 2000.

### BACKGROUND OF THE INVENTION

A well established approach for drug discovery is the utilization of a biological assay to screen a large database of proprietary compounds (>100,000) to identify initial leads that effect the activity of target protein(s) in the assay (for reviews see—J. W. Armstrong, Am. Biotechnol. Lab. 17, 26, 28 (1999); J. E. Gonzalez, P. A. Negulescu, Curr. Opin. Biotechnol. 9, 624-631 (1998); K. R. Oldenburg, Annu. Rep. Med. Chem 33, 301-311 (1998); P. B. Fernandes, Curr. Opin. Chem. Biol. 2, 597-603 (1998); B. A. Kenny, M. Bushfield, D. J. Parry-Smith, S. Fogarty, J. M. Treherne, Prog. Drug Res 51, 245-269 (1998); L. Silverman, R. Campbell, J. R. Broach, Curr. Opin. Chem. Biol. 2, 397-403 (1998). The resulting identification of lead chemical compounds from the high-throughput screening (HTS) effort initiates an iterative approach to optimizing the activity of the small molecules from feedback obtained from structural and biological activity data. A major drawback of this method is the typical requirement that the biological assay be completely re-designed with the identification of each new protein target. This effectively requires a large commitment of resources and time before new drug discovery projects can be initiated. Besides the difficulty associated with the design of a biological assay to properly screen the chemical library for the desired activity, there exists a number of other limitations that may hinder the analysis and utility of the assay. These are usually a result of the necessary complexity of the assay to reasonably mimic the cellular function of the target protein and to monitor changes in its activity. It is not uncommon for a biological assay to contain multiple proteins, to be a membrane based assay, or to even be a cell based assay. The consequence of a complex assay is the ambiguous nature of a positive hit since details of the chemical interaction between a target protein and small molecule is not readily correlated to an observed biological response. As a result, these assays greatly limit a structure based approach to drug optimization while making it extremely difficult to decipher a structure-activity relationship (SAR) from the initial chemical leads.

NMR has been extensively used to evaluate ligand binding with an obvious utility in structure based drug design (K. Wuthrich, NMR of Proteins and Nucleic Acids (John Wiley & Sons, Inc., New York, 1986); G. Otting, Curr. Opin. Struct. Biol. 3, 760-8 (1993); P. J. Whittle, T. L. Blundell, Annu. Rev. Biophys. Biomol. Struct 23, 349-75 (1994); T. L. Blundell, Nature 384, Suppl.), 23-26 (1996)). The “SAR by NMR” method previously described by Hajduk et al. illustrates this utility of NMR to screen small molecules for their ability to bind proteins from observed chemical shift perturbation in a 2D ¹H—¹⁵N-HSQC spectrum (P. J. Hajduk, et al., J. Med. Chem. 40, 3144-3150 (1997); P. J. Hajduk, et al., J. Am. Chem. Soc. 119, 5818-5827 (1997); S. B. Shuker, P. J. Hajduk, R. P. Meadows, S. W. Fesik, Science 274, 1531-1534 (1996)). In addition to determining if the small molecule binds the protein, the observed chemical shift perturbations also allow for the identification of the binding site of the protein. The concept of using NMR as a primary screen has some significant obstacles that may limit its use in a high-throughput format. Mainly, the relatively low sensitivity of NMR requires significant quantities of isotope enriched protein (>0.2 mM) and data acquisition time (>10 minutes) per sample which drastically impacts the number of compounds that can be screened (L. E. Kay, P. Keifer, T. Saarinen, J. Am. Chem. Soc. 114, 10663-5 (1992); J. Schleucher, et al., J. Biomol. NMR 4, 301-6 (1994)). A response to these problems has been the utilization of mixtures, but this then requires deconvolution of the positive hits which incurs a further commitment of sample supply and instrument resources. Furthermore, the utilization of mixtures may limit a compound's solubility below the concentration required by NMR while further complicating the necessity of maintaining consistent buffer conditions (pH, ionic strength) between samples. Additionally, the need to optimize the NMR data collection throughput usually results in a compromise between data quality and acquisition time.

Other attempts to minimize resource and sample requirements have focused on the application of 1D NMR techniques, particularly diffusion-edited measurements and transfer NOEs (M. J. Shapiro, J. R. Wareing, Curr. Opin. Drug Discovery Dev. 2, 396-400 (1999); B. Meyer, T. Weimar, T. Peters, Eur. J. Biochem. 246, 705-709 (1997); M. Lin, M. J. Shapiro, J. R. Wareing, J. Am. Chem. Soc. 119, 5249-5250 (1997); M. Lin, M. J. Shapiro, J. R. Wareing, J. Org. Chem. 62, 8930-8931 (1997); P. J. Hajduk, E. T. Olejniczak, S. W. Fesik, J. Am. Chem. Soc. 119, 12257-12261 (1997)). The 1D NMR experiments eliminate the need for labeled protein while minimizing sample quantities and data acquisition time. Unfortunately, the ID NMR experiments do not provide information on the location of the binding site. They also have a lower sensitivity to weak binders compared to the 2D ¹H—¹⁵N-HSQC experiments while requiring a more complicated method for automated data analysis. Additionally, the utilization of mixtures is more difficult because of spectral overlap. Recently developed NMR cryoprobes and flow-through probes may provide some solutions to these issues since they may provide a 3-4 fold increase in sensitivity and a method of increase throughput, respectively (M. J. Shapiro, J. R. Wareing, Curr. Opin. Drug Discovery Dev. 2, 396-400 (1999)). Nevertheless, NMR may not be ideal for the initial stage of the screening process since typical NMR experiments are time consuming and resource intensive. Given the observation that most assays have a hit rate on the order of 0.1 to 1% which means that >99% of the data collected is negative information, it appears to be a more logical approach to eliminate a majority of the compounds before the NMR analysis stage.

A new, rapid approach to drug design is provided by the present invention and provides the details useful for structure based drug design, combined with the capability to screen very small quantities of multiple compounds rapidly and accurately.

### SUMMARY OF THE INVENTION

The present invention provides a method of screening a compound mixture to identify compounds which bind to a target molecule by preparing a mixture of compounds, each compound having a known molecular weight, and incubating the mixture with target molecule to allow formation of bound compound-target complex. Mass spectral analysis is performed to determine the identity of bound compound based upon molecular weight. A complex of identified compound bound to target molecule is prepared and the NMR chemical shift perturbation of the complex of identified compound bound to target molecule is analyzed to identify the location of the binding site of compound on target molecule. Using the NMR data, a molecular model can be prepared and computer assisted drug design can be used to design high affinity ligands for the target molecule.

The present invention also provides methods of designing a ligand having improved affinity for a target molecule comprising preparing a mixture of compounds having known molecular weights and incubating the mixture with target molecule to allow formation of bound compound-target complex. The compound-target complex is separated from unbound compound and mass spectral analysis is performed on compound-target complex to determine the identity of bound compound based upon molecular weight. A complex of identified compound bound to target molecule is prepared and NMR is performed. The NMR shift perturbation of the complex of identified compound bound to target molecule is analyzed to identify the binding site of the compound on the target molecule and a library of structural analogs having known molecular weights is designed based upon the chemical structure of the identified compound and the identified binding site of the target molecule. The library of structural analogs is prepared and binding of the structural analogs to the target molecule is determined.

Further in accordance with the present invention is provided a method of designing a high affinity ligand for a target molecule by preparing a mixture of compounds, each compound having a known molecular weight, and incubating the mixture with target molecule to allow formation of bound compound-target complex. Mass spectral analysis is performed to identify bound compound. Complexes of identified compounds bound to target molecule are prepared and the NMR shift perturbation of complexes of identified compound bound to target molecule are analyzed to identify at least two compounds having at least two different binding sites on the target molecule. The spatial orientation of the compounds on the target molecule is determined and the structural information of the at least two identified compounds are used to design a ligand which binds at the identified sites and minimally affects the determined spatial orientation. Linking may be by molecular modeling or by chemical linkage.

### BRIEF DESCRIPTION OF THE FIGURES

FIG. 1 is a ESI mass spectral analysis of filtrate after passing MMP-1 inhibitors through Sephedex G-25 columns in the presence and absence of MMP-1. (A) 45 μM compound 1 (MW 393) and 45 μM MMP-1, (B) 45 μM compound 1 alone, (C) 250 μM compound 2 (MW 457) and 50 μM MMP-1, (D) 250 μM compound 2 alone, (E) 8 mM compound 3 (MW 394) and 0.4 mM MMP-1, (F) 8 mM compound 3 alone.

FIG. 2 is an ESI (positive ionization) mass spectral analysis of the filtrate from the gel-filtration titration of compound 2 (MW 457) with MMP-1 (A) MMP-1 alone at 50 μM; (B-E) increasing amount of MMP-1 (B) 20 μM, (C) 30 μM, (D) 40 μM and (E) 50 μM and increasing amount of compound 2 from (B) 100 μM, (C) 150 μM, (D) 200 μM and (E) 250 μM; and (F) 250 μM compound 2 alone.

FIG. 3 is an ESI (negative ionization) mass spectral analysis of the filtrate from the gel-filtration analysis of a mixture containing 1 mM each of ten known MMP-1 inhibitors (TOP) with 0.1 mM MMP-1 and (BOTTOM) without MMP-1. The mass ions for the ten compounds are highlighted on the spectrum. The mixture is composed of compounds 4-13 listed in Table 1.

FIGS. 4A-C are 2D ¹H—¹⁵N HSQC spectra of free MMP-1 (multiple contours) overlayed with MMP-1 complexed with (A) compound 1, (B) compound 2 and (C) compound 3 (1-2 contours) identified as binders from the gel-filtration/mass spectral analysis (FIG. 1).

FIG. 5 (A) A GRASP(32) surface of the NMR solution structure of MMP-1 where residues that incurred a perturbation in the ¹H—¹⁵N HSQC spectrum in the MMP-1:compound 1 complex are colored black, indicating the location of the ligand interaction with the protein.

FIG. 5(B) NMR structure of the MMP-1:compound 1 complex. Compound 1 is shown with thicker bonds.

### DETAILED DESCRIPTION OF THE INVENTION

The present invention provides a method of screening compounds to identify compounds which specifically bind to a target molecule and to identify the site of binding. This invention also provides a fast and efficient method of designing ligands for a given target molecule.

In accordance with methods of the present invention, mixtures of ligands or compounds such as small molecules are prepared. The ligands may be for example, from commercial sources, from preexisting chemical libraries, or prepared according to need, such as based upon previous structure activity relationship information. Each mixture is comprised of a group of ligands, each having a known molecular weight. In some preferred embodiments of the present invention each ligand has a unique molecular weight which preferably differs from other ligands of the mixture by more than 3Da to allow for clear identification of each component. In some aspects of the invention, the molecular weight of each ligand is preferably less than about 2000, and where linkage of one or more compounds is anticipated, the molecular weight may be more preferably less than about 350. In addition to molecular weight, ligands may be chosen based upon, for example, acidity, reactivity, shape and functional groups of the compounds. Diversity of libraries is generally preferred. Ligand concentration will vary depending upon the number of ligands forming the mixture. In general the compound mixture comprises at least about 0.1 nM of each compound to be screened, and more preferably at least about 1 mM of each compound.

The compound mixture is incubated with a target molecule (such as a protein, nucleic acid, etc.). Target molecule may be obtained from commercial sources, may be purified from natural sources or may be prepared recombinantly. In general, the incubation mixture contains at least about 10 μM of target molecule and preferably from about 50 μM to about 200 μM and most preferably about 100 μM.

Complexes of bound compound-target molecule are separated from unbound compounds by running the mixture through a size exclusion column such as by suction filtration or centrifugation. Such chromatography techniques such as gel permeation chromatography (GPC) spin column are described in J. Mass. Spect. 33: 264-273 (1998) which is incorporated by reference herein. Size exclusion chromatography is based upon the premise that low molecular weight compounds are retained in the column, and high molecular weight compounds are passed through the column. Thus, compounds which elute from the column should have bound to the target molecule and are thus highly likely to be active in a biological assay involving the target.

A compound from the mixture may be easily identified once bound to a target molecule, on the basis of its molecular weight as determined by mass spectrometry which is performed on the filtrate in the molecular weight range for the compounds in the mixture. Since the molecular weights are known for each compound in the mixture, the observation of an ion peak in the mass spectrometer simultaneously identifies the presence of a hit and the compound identity. In preferred embodiments of the present invention, each of the compounds of the mixture has a unique molecular weight. A target-specific assay to identify candidates from a mixture is avoided allowing for easy automation. In addition, deconvolution is generally avoided. Where deconvolution is necessary such as when the molecular weight of a hit corresponds to more than one compound of the mixture or fragment thereof, it is generally of limited scope and can be rapidly carried out.

In some preferred embodiments of the present invention, the size exclusion column can be prepared with any size-exclusion resin such as Sephadex G25 resin (Pharmacia) that allows large molecular weight compounds to pass through the column while retaining smaller molecular weight compounds (such as those less than 2000 MW). The resin can be packed into individual columns prepared with, for instance, disposable syringes or, more preferably a 96-well filtration plate containing a low-protein-binding filter such as hydrophilic durapore filter or silanized glasswool. The small column length of the 96-well plates minimizes sample requirements and because of the high-sensitivity of MS only picomoles of the target protein are required for each sample. The protein-compound mixture can be loaded onto the size-exclusion column under a number of conditions, where the buffer conditions, number of compounds in the mixture and the protein-compound molar ratios may be varied. The filtrate from the column is collected in a standard 96-well plate by either centrifugation or suction filtration of the resin-filled 96-well filtration plate. The technique is sensitive to weak protein-drug interactions.

Mass spectral analysis may be performed on the mixture without separating bound and unbound compound. Mass spectral analysis is performed such as with electrospray ionization (ESI) MS methods in both positive and negative ionization modes. Background noise is differentiated from unique molecular ion peaks and the molecular weight leading to the identity of bound compounds, is determined based upon the difference between the weight of the target molecule and the weight of any complex which correlates to a peak corresponding to a unique chemical entity. Alternatively, matrix assisted laser desorption/ionization MALDI/MS can be used.

These steps can be easily automated using robotics. For instance, a Gilson 215 liquid handler may be used to transfer the filtrate from the 96-well plates to the mass spectrometer.

Once the identity of a compound (ligand) which binds to a target is known, the specific binding site may be determined using NMR spectroscopy, for instance, by mapping NMR chemical shift perturbations onto the structure of the target. The three dimensional structure of the target may be obtained from standard X-ray, NMR or homology modeling techniques and the NMR resonance assignments from standard NMR protocols. The chemical shift perturbations may be obtained by comparing the NMR spectra of the free target with the NMR spectra of the target complexed with the identified ligand, where the NMR spectra may correspond to standard 2D ¹H—¹⁵N HSQC, 2D ¹H—¹³C HSQC, 2D ¹H—¹⁵N HMQC or 2D ¹H—¹³C HMQC experiments using either ¹⁵N-enriched or ¹³C/¹⁵N-enriched proteins or targets. The observed NMR resonances for the target that exhibit a chemical shift perturbations in the presence of the ligand are assigned to a residue in the target by utilizing the NMR resonance assignments for the free target. The residues in the target that experience chemical shift perturbations in the presence of the ligand are then mapped onto the structure of the target to define the binding site of the ligand on the target. Any enriched target molecule may be used, and preferably polypeptides serve as the target. The target molecule can be labeled with ¹³C or ¹⁵N using methods known in the art. In preferred embodiments the target molecule is prepared in recombinant form using transformed host cells. The “SAR by NMR” procedure utilizing NMR-chemical shift perturbation and linking of molecular fragments for drug design has been disclosed in international patent application publication Number WO 97/18469 and WO 97/18471; and published in Science 274:1531-1534 (1996); JACS 119:5818-5827 (1997) and J. Med. Chem.40:3144-3150 (1997).

A preferred means of preparing adequate quantities of uniformly labeled polypeptides is to transform a host cell with an expression vector that contains a polynucleotide that encodes the polypeptide and culture the transformed cells in a medium that contains assimilable sources of radiolabel. Such sources are well known in the art. For instance, ¹⁵NH₄Cl, ¹³C Glucose or (¹⁵NH₄)₂SO₄ may be used.

Means for preparing expression vectors that contain polynucleotides encoding specific polypeptides are well known in the art, as are means for transforming host cells with vectors and culturing those transformed cells so that the polypeptide is expressed.

Given the protein and compound structure and the general location of the compound binding site from the NMR chemical shift perturbations, standard modeling techniques are applied to define a computer model of the complex. The resulting computer model of the complex may be verified by consistency between predicted short (<5 Å) hydrogen pair distances and NOEs observed in NMR spectra of the complex and/or X-ray structures of the complex.

The affinity of the compound for the protein (K_{d} and/or IC50) can be determined from a variety of accepted techniques which may include K_{d} measurements from NMR diffusion coefficient changes or chemical shift perturbations and/or IC50 determination from a specific biological assay for the protein target to determine biological relevance of the hit.

If more than one ligand having a unique binding site is identified, the three dimensional structure and spatial orientation of the ligands in relation to the target, as well as in relation to each other may be determined. Spatial orientation of each ligand to the target molecule allows for identification of portions of the ligand which are in close proximity to the atoms in the target, as well as portions which are distal from atoms in the binding site and which may be involved in interactions with other molecules in situ.

Once the specific binding site has been identified, three dimensional models may be generated using any one of a number of methods known in the art, and include, but are not limited to, homology modeling as well as computer analysis of raw structural coordinate data generated using crystallographic or spectroscopy techniques. Computer programs used to generate such three dimensional models and/or perform the necessary fitting analysis include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Franscisco, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis, Mo.), and LEAPFROG (TRIPS, Inc., St. Louis, Mo.).

These and other computer programs will be well known to those of ordinary skill in the art. Once the relevant data has been analyzed by such programs, candidate ligands can be identified, prepared and tested for their ability to bind to a target and for its biological activity.

Identified ligands which bind to the target molecule may then be tested in biological systems to confirm that biological activity correlates with the observed binding. In traditional systems, IC50 values are obtained for each ligand from the biological assay that provides an initial ranking of the effectiveness of the chemical leads. As a follow up Kd values might be obtained from NMR titration data or a variety of other analytical techniques. The present invention inverts these typical steps, thereby eliminating the need to convert a standard biological assay to a high throughput format. Rather, the number of leads is reduced so that the standard assay need not be converted.

Following verification of biological activity, a refined structure of the protein-ligand complex may be elucidated by NMR, X-ray and/or modeling.

Further, a library of structural analogs may be prepared based upon the initial lead or leads, and tested for binding in accordance with the present invention, thereby further optimizing the affinity and activity of the ligand. For instance, a lead compound may be derivatized at one or more positions in the molecule based upon points of interaction at the binding site in accordance with known chemical principals to provide structural analogs. Combinatorial syntheses may be particularly useful for these purposes. In addition, where more than one ligand having unique binding sites is identified, the spatial orientation of the ligands with the binding site can be used to design new high affinity ligands. New ligands can be designed by modeling techniques or by chemical linkage of two compounds. In this way two or more compounds having a given affinity for a target may be linked resulting in a compound with improved affinity for a target. The design of a linker is based on the distances and angular orientation needed to maintain each of the ligands in proper orientation to the target. Suitable linkers are well known and can easily be identified by those skilled in the art. J. of Computer Aided Molecular Design 6:61-78 (1992), Perspectives in Drug Discovery and Design 3:21-33 (1995), J. Med. Chem. 27(5), 557-563 (1984), Science 263:380-384 (1994).

The following examples are meant to illustrative the effectiveness of methods of the present invention by employing compounds previously tested against a given target, MMP-1. The examples are not meant to be limiting of the present invention.

### EXAMPLES

### Example 1

Compound having known affinities for MMP-1 were chosen. The compounds are provided in Table 1.

TABLE 1
Inhibitors of MMP-1.

| Compound | | IC50 | |
|---|---|---|---|
| Number | Chemical Name | (nM) | MW |
| 1 | N2-(4-Methoxy-benzenesulfonyl)- | 9 | 393 |
| | N2-[(pyridin-3-yl)methyl]- | | |
| | N-hydroxy-D-valinamide | | |
| 2 | N-Hydroxy-2-[(4-methoxy- | 9900 | 457 |
| | benzenesulfonyl)-pyridin-3-ylmethyl- | | |
| | amino]-isophthalamic acid | | |
| 3 | [(2-Hydroxycarbamoyl-6-methyl-phenyl)- | 89000 | 394 |
| | (4-methoxy-benzenesulfonyl)-amino)- | | |
| | acetic acid | | |
| 4 | 2-[Benzyl-(4-methoxy-benzenesulfonyl)- | 408 | 426 |
| | amino]-N-hydroxy-5-methyl-benzamide | | |
| 5 | 8-Methoxy-4-[(4-methoxy- | 46 | 494 |
| | benzenesulfonyl)-pyridin-3-ylmethyl- | | |
| | amino]-quinoline-3-carboxylic acid | | |
| | hydroxyamide | | |
| 6 | N-Hydroxy-2-(4-methoxy- | 139 | 399 |
| | benzenesulfonyl)-2-methyl-3-naphthalen- | | |
| | 2-yl-propionamide | | |
| 7 | 2-(4-Methoxy-benzenesulfonyl)-5-methyl- | 760 | 407 |
| | 2-pyridin-3-ylmethyl-hexanoic acid | | |
| | hydroxyamide | | |
| 8 | 4-(Methyl-[4-(pyridin-4-yloxy)-benzene- | 1012 | 450 |
| | sulfonyl]-amino)-quinoline-3-carboxylic | | |
| | acid hydroxyamide | | |
| 9 | 1-(Furan-2-carbonyl)-4-(4- | 17 | 471 |
| | methoxy-benzene-sulfonyl)-2,3,4,5- | | |
| | tetrahydro-1H-[1,4]-benzodiazepine-3- | | |
| | carboxylic acid hydroxyamide | | |
| 10 | 4-(4-Butoxy-benzenesulfonyl)-1-methyl- | 3417 | 370 |
| | piperidine-4-carboxylic acid hydroxya- | | |
| | mide | | |
| 11 | 5-Bromo-N-hydroxy-3-methyl-2-[methyl- | 1095 | 449 |
| | (naphthalene-2-sulfonyl)-amino]- | | |
| | benzamide | | |
| 12 | 4-(4-Butoxy-benzenesulfonyl)-1-ethyl- | 7062 | 384 |
| | piperdine-4-carboxylic acid hydroxyamide | | |
| 13 | 3-[4-(2-Azepan-1-yl-ethoxy)-phenyl]- | 540 | 491 |
| | N-hydroxy-2-(4-methoxy- | | |
| | benzenesulfonyl)-2-propionamide | | |

### Example 2

Protein-single Compound Incubation

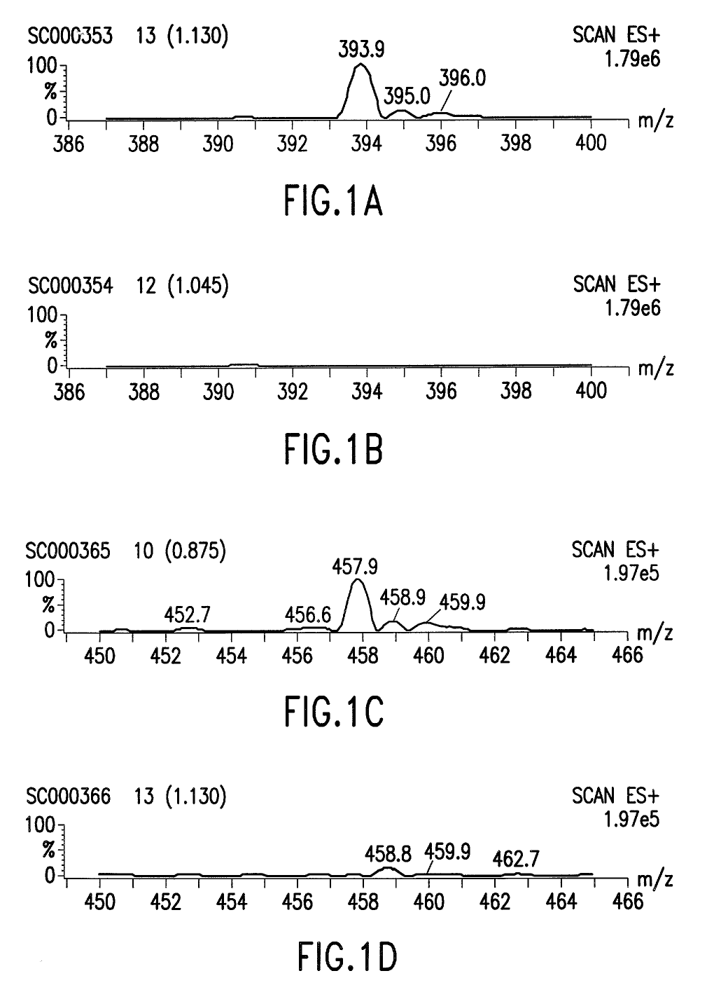

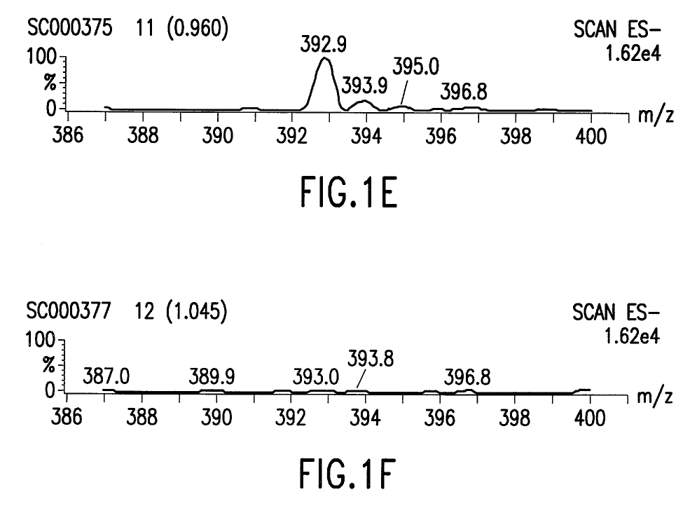

1 mM each of compounds 1, 2 and 3 (Table 1) were dissolved in DMSO and each incubated alone or in the presence of MMP-1 at a 0.1 mM in a buffer consisting of 20 mM Tris, 100 mM NaCl, 5 mM CaCl₂, 0.1 mM ZnCl₂, 2 mM NaN₃ and 3.5 mM DTT at pH 6.5 at room temperature for 30 minutes. The final concentration of DMSO in the MMP-1:compound mixture was 5%. A total volume of 25 μl of each sample was loaded on a Sephedex G25 column in a Millipore multiscreen filtration system composed of a 0.65 μm hydrophilic durapore filter. The samples were eluted using centrifugation (15,000×g for 3 minutes). Samples were collected and analyzed by mass spectroscopy using automated ESI/MS methods in both positive and negative ionization modes with a Micromass LCT quadrapole time of flight mass spectrometer and Quattro I triple-quadrapole mass spectrometer each equipped with a Gilson 215 liquid handler. Results in FIG. 1 show that unbound compound is retained (FIGS. 1B (Compound 1), 1D (Compound 2) and 1F (Compound 3)), while compound bound to MMP-1 is eluted (FIGS. 1A (Compound 1+MMP-1), 1C (Compound 2+MMP-1) and 1E (Compound 3+MMP-1)).

### Example 3

Titration

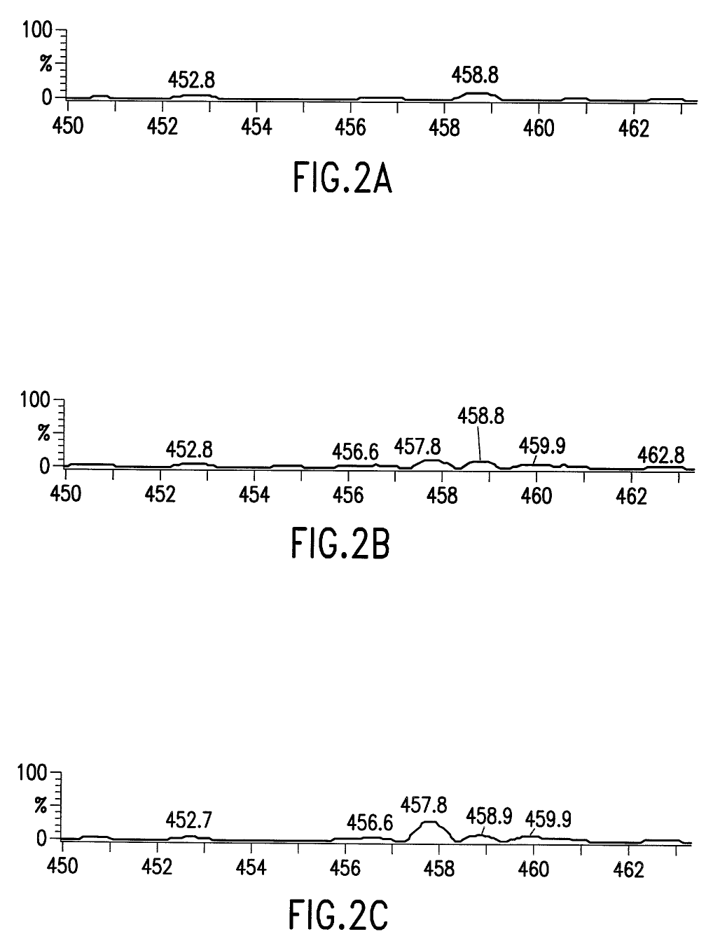

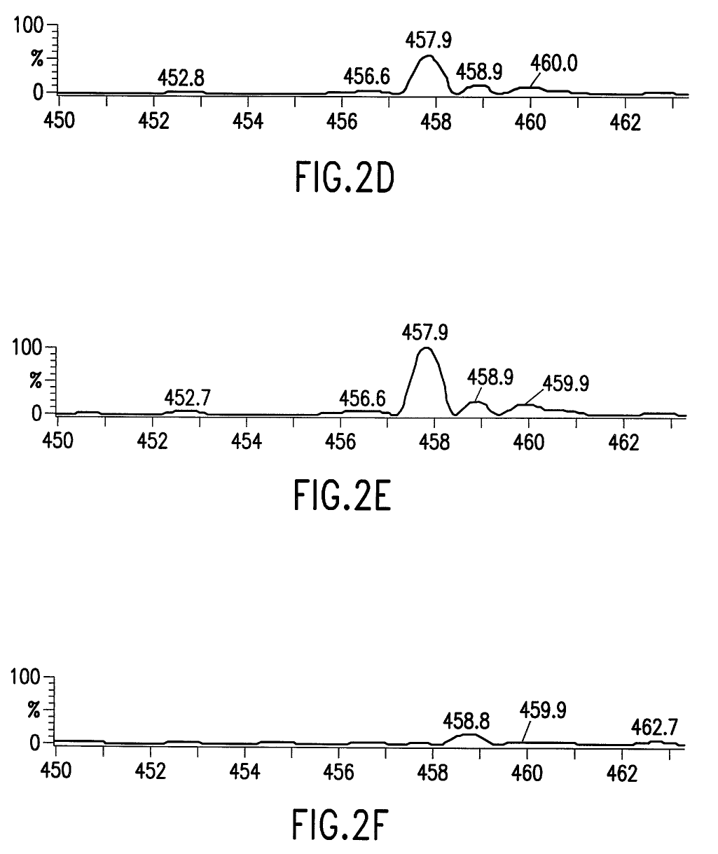

Increasing amounts of Compound 2 were incubated alone or with increasing amounts of MMP-1 as described in Example 2. FIG. 2A-E provide ESI (positive ionization) mass spectral analysis of these filtrates.

| FIG. | Compound | MMP-1 |
|---|---|---|
| 2A | 0 | 50 |
| 2B | 100 uM | 20 uM |
| 2C | 150 uM | 30 uM |
| 2D | 200 uM | 40 uM |
| 2E | 250 uM | 50 uM |
| 2F | 250 uM | 0 |

FIG. 2 shows that the relative intensity of the [M+H]¹⁺ (m/z) 457.9 ion correlates with the increase in MMP-1 concentration.

### Example 4

Protein-mixture Incubation

A mixture of ten compounds described in Example 1 are provided at an approximate concentration of 1 mM each was dissolved in DMSO. The ligand mixture was incubated alone or with MMP-1 at a concentration of 0.1 mM in a buffer consisting of 20 mM Tris, 100 mM NaCl, 5 mM CaCl₂, 0.1 mM ZnCl₂, 2 mM NaN₃ and 3.5 mM DTT at pH 6.5 at room temperature for 30 minutes. The final concentration of DMSO in the MMP-1 :compound mixture was 5%.

### Example 5

Gel Filtration/Mass Spectroscopy Collection of Samples

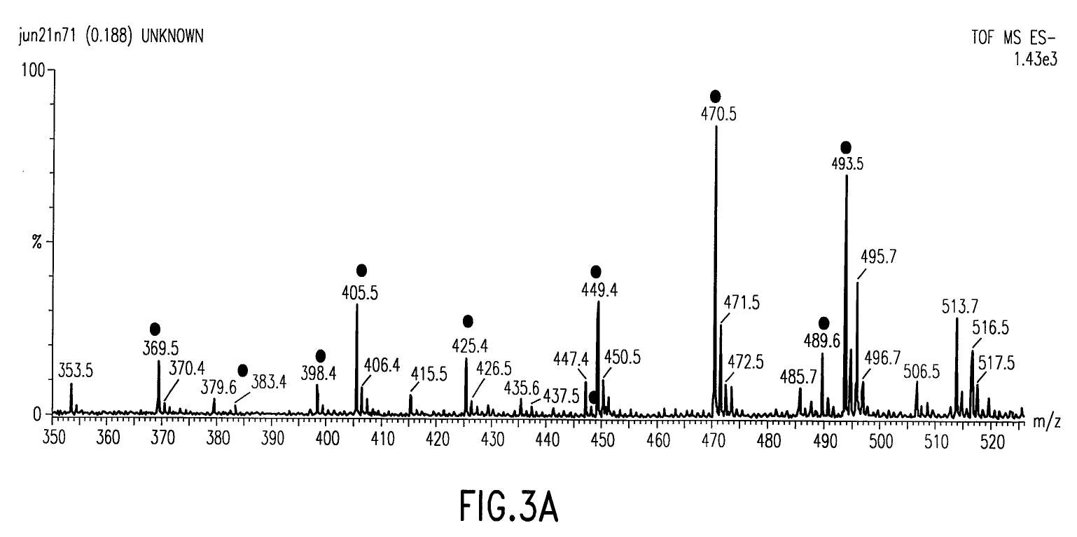

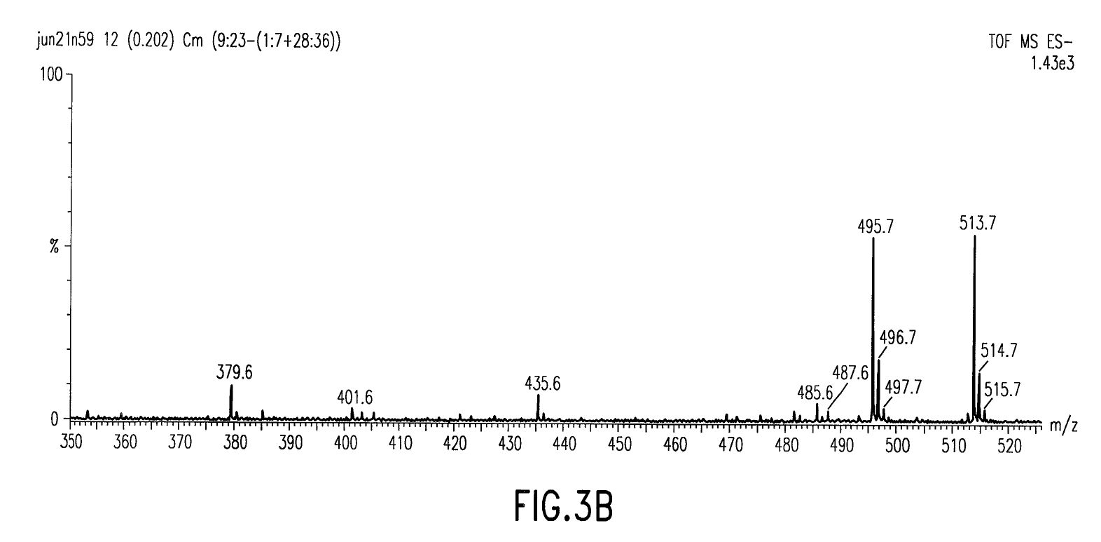

A total volume of 25 μl of the MMP-1-compound mixture is loaded on a Sephedex G25 column in a Millipore multiscreen filtration system composed of a 0.65 μm hydrophilic durapore filter. The samples were eluted using centrifugation (15,000×g for 3 minutes). Samples were collected and analyzed by mass spectroscopy using automated ESI/MS methods in both positive and negative ionization modes with a Micromass LCT quadrapole time of flight mass spectrometer and Quattro I triple-quadrupole mass spectrometer each equipped with a Gilson 215 liquid handler. Results are shown in FIGS. 3A (with MMP-1) and B (without MMP-1). Mass ions for the ten compounds are highlighted on the spectra.

### Example 6

NMR Analysis of MS Hits

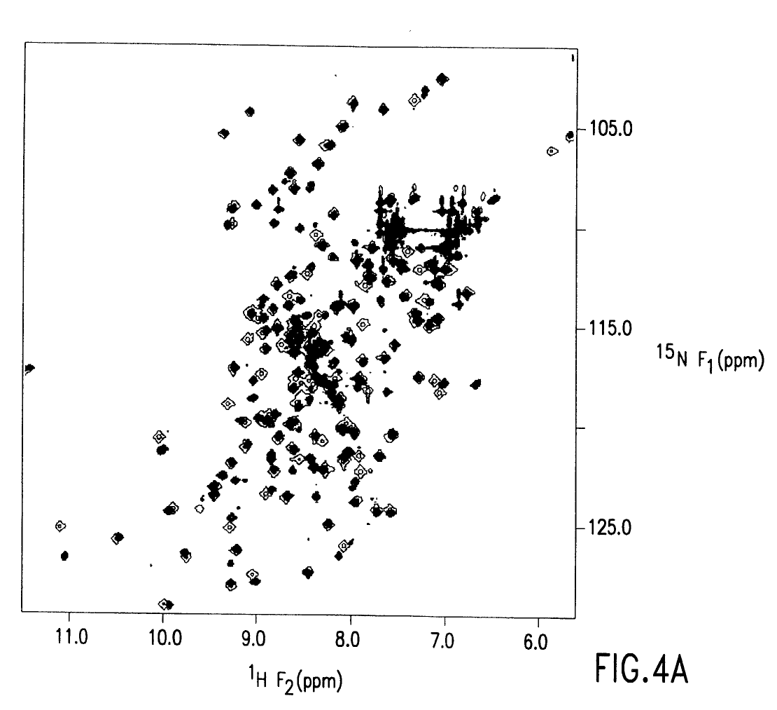

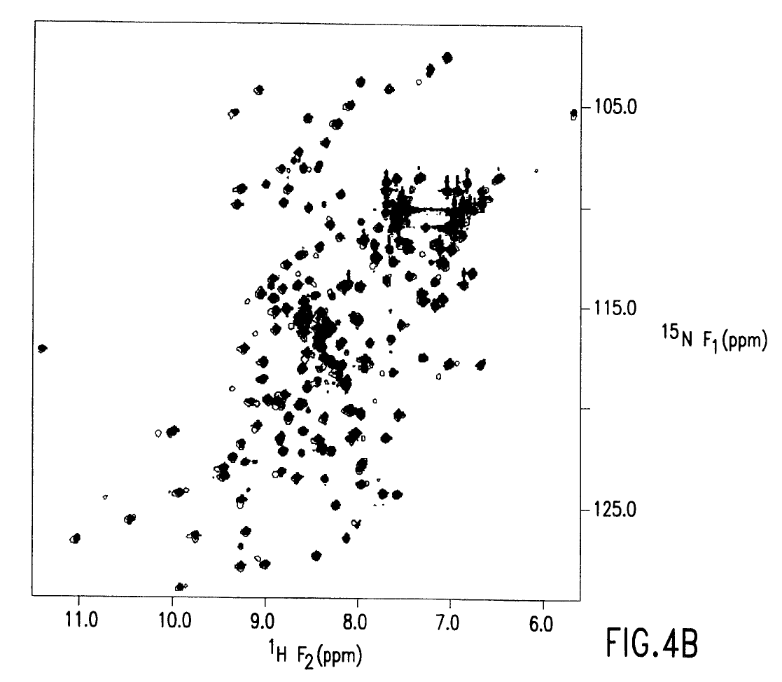

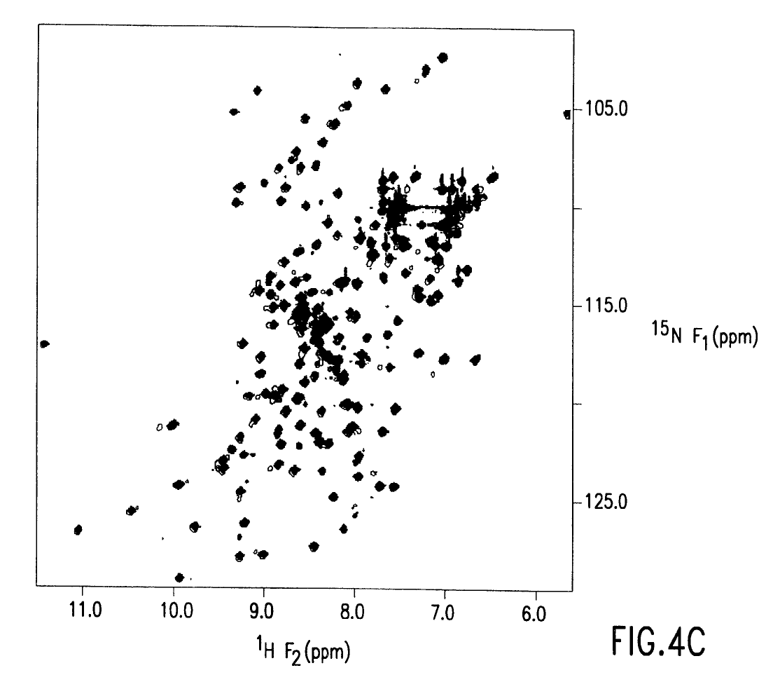

MMP-1 was labeled as described in Moy, J. Biomol. NMR, Vol. 10: 9-19 (1997). Compounds 1, 2 and 3 were selected from Example 5. The gradient enhanced 2D ¹H—¹⁵N HSQC spectra were collected on a 0.2 mM ¹⁵N-MMP-1 in a buffer consisting of 20 mM Tris, 100 mM NaCl, 5 mM CaCl₂, 0.1 mM ZnCl₂, 2 mM NaN₃ and 3.5 mM DTT in 90% H₂O and 10% D₂O at pH 6.5 and 35° C. with compounds titrated to achieve concentrations of compound 1, 2 and 3 ranged from 0.2-4.0 mM. The 2D ¹H—¹⁵N HSQC spectra were recorded with 256 complex points in t1, 2048 real points in t2, and 192 scans per increment. Spectra windows for t1 and t2 were 1723.7 and 8064.5 Hz, respectively, with the carrier at 4.75 and 115.2 ppm, respectively. Data were processed and analyzed using NMRPipe, NMRWish [F. Delaglio, S. Grzesiek, G. W. Vuister, G. Zhu, J. Pfeifer, and A. Bax J. Biomol. NMR 6, 277 (1995). ] and PIPP [D. S. Garrett, R. Powers, A. M. Gronenborn, and G. M. Clore J. Magn. Reson. 95, 214-20 (1991).] on a Sun Ultra 10 workstation. FIG. 4 provides the spectra of free MMP-1 (multiple contours) overlayed with MMP-1 complexed with Compound 1 (FIG. 4A), Compound 2 (FIG. 4B) and Compound 3 (FIG. 4C) (1-2 contours). All three compounds induce chemical shift perturbations for residues in the vicinity of the catalytic Zn and S1′ pocket in the MMP-1 active site. Particularly, residues 80-83, 114-119 and 136-142 exhibited the largest chemical shift changes in the presence of the inhibitors. The extent of the chemical shift perturbations and the number of residues exhibiting the chemical shift change is directly related to the observed IC50 for each of the compounds. (FIG. 4A, 4B, 4C), i.e. stronger binding contributes to greater perturbations and weaker binding to less perturbations.

### Example 7

Modeling

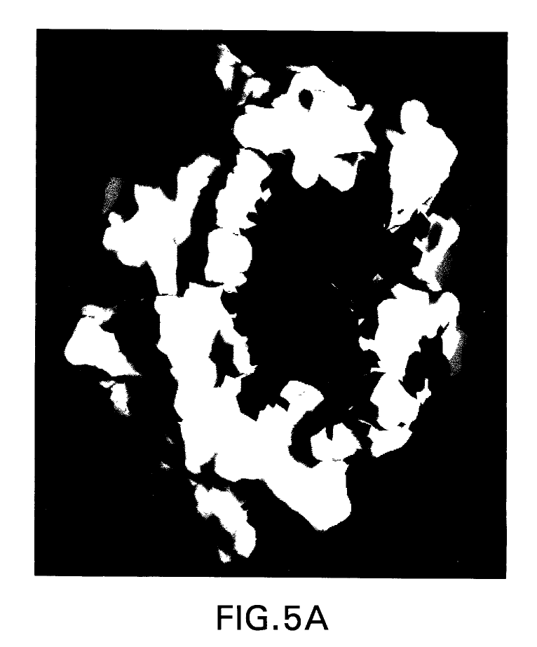

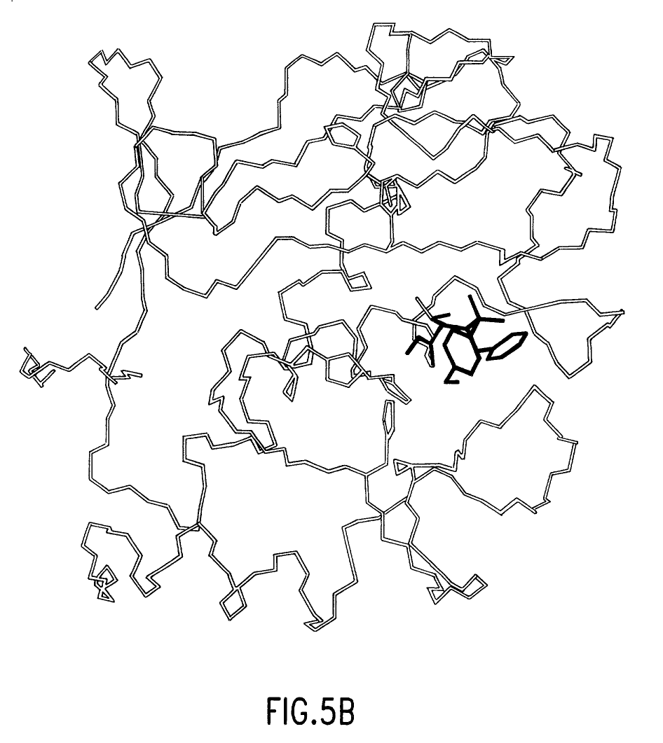

Using computer modeling, a GRASP surface of the NMR solution structure of MMP-1 was designed (FIG. 5A) where residues that incurred a perturbation in the spectra from Example 7 in the MMP-1:Compound 1 complex are colored blue, indicating the location of the ligand interaction with the protein. An NMR structure is designed of the MMP-1:Compound 1 complex. (FIG. 5B). Compound 1 is shown with thicker bonds.

What is claimed is:

1. A method of screening a compound mixture to identify the binding site of a compound which binds to a target molecule comprising:
a) preparing a mixture of compounds of known molecular weights;
b) incubating the mixture with target molecule to allow formation of bound compound-target complex;
c) performing mass spectral analysis on compound-target complex to determine the identity of bound compound based upon molecular weight;
d) preparing a complex of identified compound bound to target molecule; and
e) analyzing the NMR chemical shift perturbations of the complex of identified compound bound to target molecule to identify the location of the binding site of compound on the target molecule.

2. The method of claim 1 further comprising separating the compound-target complex from unbound compound.

3. The method of claim 2 wherein the compound-target molecule complex is separated from unbound compound using a size exclusion column.

4. The method of claim 2 wherein the compound-target molecule complex is separated from unbound compound using a multiscreen filtration system packed with size exclusion gel.

5. The method of claim 1 wherein each compound has a molecular weight of less than about 2000 MW.

6. The method of claim 1 further comprising testing the identified compound for biological activity against the target molecule.

7. The method of claim 1 further comprising preparing a molecular model of the complex.

8. The method of claim 7 further comprising designing a ligand with improved affinity for the target molecule using computer-assisted rational drug design.

9. The method of claim 7 wherein the molecular model is determined using one or both of NMR and X-ray crystallographic data.

10. A method of designing a ligand having improved affinity for a target molecule comprising:
a) preparing a mixture of compounds having known molecular weights;
b) incubating the mixture with target molecule to allow formation of bound compound-target complex;
c) performing mass spectral analysis on compound-target complex to determine the identity of bound compound based upon molecular weight;
d) preparing a complex of identified compound bound to target molecule;
e) analyzing the NMR shift perturbations of the complex of identified compound bound to target molecule to identify the binding site of the compound on the target molecule;
f) designing a library of structural analogs having known molecular weights based upon the chemical structure of the identified compound and the identified binding site of the target molecule;
g) synthesizing said structural analogs; and
h) determining whether the structural analogs binds to the target molecule.

11. The method of claim 10 further comprising testing the structural analogs for biological activity against the inhibitor.

12. The method of claim 10 wherein the structural analogs are tested for binding by
a) incubating the structural analogs with target molecule to allow formation of bound structural analog-target complex;
b) performing mass spectral analysis on structural analog-target complex to determine the identity of bound structural analog based upon molecular weight;
c) preparing a complex of identified structural analog bound to target molecule; and
d) analyzing the NMR chemical shift perturbations of the complex of structural analog bound to target molecule to identify the location of the binding site of compound on the target molecule.

13. The method of claim 10 further comprising separating the compound-target complex from unbound compound.

14. The method of claim 13 wherein the compound-target molecule complex is separated from unbound compound using a size exclusion column.

15. The method of claim 13 wherein the compound-target molecule complex is separated from unbound compound using a multiscreen filtration system packed with size exclusion gel.

16. The method of claim 10 wherein each compound has a molecular weight of less than about 2000 MW.

17. The method of claim 10 further comprising preparing a molecular model of the complex.

18. The method of claim 17 wherein the molecular model is prepared using NMR and X-ray crystallographic data.

19. The method of claim 10 further comprising designing structural analogs using computer-assisted rational drug design.

20. A method of designing a high affinity ligand for a target molecule comprising:
a) preparing a mixture of compounds having known molecular weights;
b) incubating the mixture with target molecule to allow formation of bound compound-target complex;
c) performing mass spectral analysis on compound-target complex to determine the identity of bound compound based upon molecular weight;
d) preparing complexes of identified compounds bound to target molecule;
e) analyzing the NMR shift perturbations of complexes of identified compound bound to target molecule to identify at least two compounds having at least two different binding sites on the target molecule; and
f) determining the spatial orientation of the compounds on the target molecule;
g) linking at least two identified compounds to minimally affect the determined spatial orientation.

21. The method of claim 20 wherein the at least two identified compounds are linked using molecular modeling.

22. The method of claim 20 wherein the compound-target complex and unbound compounds are separated.

23. The method of claim 22 wherein the compound-target complex and unbound compounds are separated using size exclusion column chromatography.

24. The method of claim 22 wherein the compound-target molecule complex is separated from unbound compound using a multiwell filtration system packed with size exclusion gel.

25. The method of claim 20 wherein each compound has a molecular weight of less than about 2000 MW.
